(19) United States
(12) Patent Application Publication
(10) Pub. No.: US 2024/0425814 A1
(43) Pub. Date: Dec. 26, 2024

(54) SALTS OF DIPEPTIDES AND THEIR USES IN CELL CULTURE

(71) Applicant: Evonik Operations GmbH, Essen (DE)

(72) Inventors: Susanne Bähr, Bad Saeckingen / Nieder-Roden (DE); Anne Benedikt, Frankfurt (DE); Christina Jost, Alsbach-Haehnlein (DE); Martin Schilling, Bonn (DE)

(73) Assignee: Evonik Operations GmbH, Essen (DE)

(21) Appl. No.: 18/698,415

(22) PCT Filed: Sep. 30, 2022
(86) PCT No.: PCT/EP2022/077368

§ 371 Date: Apr. 4, 2024

(30) Foreign Application Priority Data
Oct. 5, 2021 (EP) 21200872.6

(51) Int. Cl. C12N 5/0775; C07K 5/068; C12N 5/00

(52) U.S. Cl. CPC C12N 5/0662; C07K 5/06086; C12N 5/00; C12N 2501/998

(57) ABSTRACT

Salts of dipeptides can be used in cell cultures. A culture medium can be used for culturing cells, preferably plant cells, animal cells or mammalian cells and a method of manufacturing a cell culture product. The dipeptide salt has at least one basic amino acid and a chloride-counterion. The molar ratio of the basic amino acid to chloride-ion is between 0.8 and 1.2

### FIELD OF THE INVENTION

The present invention relates to salts of dipeptides and their uses in cell culture. Moreover, the present invention relates to biotechnological production processes. More specifically, the present invention relates to improved culture media for use in biotechnological production processes, processes employing such improved media, and to products obtained from the processes using the improved culture media.

### BACKGROUND OF THE INVENTION

Chemically defined dipeptides are widely used highly soluble and stable precursors of amino acids in the formulation of cell culture media. To increase solubility of certain limiting amino acids such as L-tyrosine or L-cystine, highly soluble amino acids such as glycine, L-alanine, L-proline and L-lysine can be coupled to form a dipeptide, which results in highly soluble, natural dipeptide precursors of those limiting amino acids. For example, WO 2011/133902 discloses cell culture media comprising dipeptides, wherein the dipeptides include amino acids having a low solubility in water, in this case tyrosine and cysteine. The authors have found that by incorporation of tyrosine and cysteine in dipeptides, solubility and stability problems of the amino acids can be ameliorated. The higher stability and solubility enable the formulation of chemically defined, highly concentrated media, that are required to run intensified and highly productive industrial cell culture processes.

WO 2012/019160 discloses animal cell cultures, wherein during the production phase the serum-free medium is supplemented with Tyr- and His-containing dipeptides. Positive effects of the addition of the Tyr- and His-containing dipeptides on growth and product formation are described.

Due to its high solubility at neutral pH, L-lysine is especially suitable as a partner for dipeptide formation to improve the solubility of amino acids with low solubility. This insight has been used to synthesize highly soluble forms of L-cystine, L-tyrosine and the branched chain amino acids L-valine, L-leucine and L-isoleucine. EP3372671 discloses Lys-containing dipeptides that have a substantially increased solubility over peptides where the lysine residue is replaced by L-alanine or L-glycine. These peptides have a beneficial effect on growth and viability of cells.

However, in addition to providing highly soluble nutrients in the form of dipeptides, there remains a need to provide these nutrients in a highly biocompatible and stable form. Highly biocompatible forms enable provision of these nutrients at high concentrations to address metabolic or bioprocessing limitations. Stability needs to be sufficient to prevent degradation in liquid or dry powder media and supplements over extended periods of time. Sufficient stability is also required to transport and store individual ingredients in dry form, ideally at ambient temperature. Especially in feed media, nutrients are present in higher concentrations. Moreover, also storage media shall contain the soluble nutrients at higher concentrations, as those media are diluted for the final application in cell culture. These points have not been sufficiently addressed and there remains a need to improve these parameters.

### SUMMARY OF THE INVENTION

The above shortcomings are addressed by the present invention. The invention is defined by the terms of the appended independent claims. Preferred embodiments of the invention are defined by the dependent claims.

Surprisingly it was found that these parameters can be improved by preparing and using the correct salt form of dipeptides that contain basic amino acids, especially for L-lysine containing dipeptides.

When preparing and comparing various hydrochloride salts with the basic, inner salt of Lys-dipeptides that does not contain an inorganic counterion, it was found that the chloride forms significantly improve storage stability of the dry powder.

It was also found that 2 HCl forms of L-lysine in the peptides are less biocompatible in high concentrations during cell culture than the 1 HCl form (and that this is not caused by a pH effect).

For example, in the case of (Lys-Cys)₂, the dihydrochloride (2 HCl) form could be provided in significantly higher concentration than the tetrahydrochloride (4 HCl) forms. In fact, while high concentration of the 2 HCl form improved cell viability, the 4 HCl form reduced cell viability at the same concentration.

The compositions according to the present invention can also be a component part of a cosmetic product, a nutritional supplement, a nutrient solution for clinical nutrition, or a cell or tissue culture medium (basal, feed or perfusion medium).

The invention further relates to the use of a culture medium of the invention for culturing cells, preferably plant cells, animal cells or mammalian cells.

Another aspect of the invention relates to a method of manufacturing a cell culture product comprising the steps of (i) providing a cell capable of producing said cell culture product; (ii) contacting said cell with a culture medium according to the invention; and (iii) obtaining said cell culture product from said culture medium or from said cell.

Preferred embodiments of the invention are described in further detail in the following detailed description of the invention.

### DETAILED DESCRIPTION OF THE INVENTION

In the context of the present invention, the expression “natural amino acids” shall be understood to include both the L-form and the D-form of the above listed 20 amino acids. The L-form, however, is preferred. In one embodiment, the term “amino acid” also includes analogues or derivatives of those amino acids.

A “free amino acid”, according to the invention, for instance “free” cysteine, is understood as being an amino acid having its amino and its (alpha-) carboxylic functional group in free form, i.e., not covalently bound to other molecules, e.g., an amino acid not forming a peptide bond. Free amino acids may also be present as salts or in hydrate form. When referring to an amino acid as a part of, or in, a dipeptide, this shall be understood as referring to that part of the respective dipeptide structure derived from the respective amino acid, according to the known mechanisms of biochemistry and peptide biosynthesis.

The present invention generally relates to dipeptide salts comprising a dipeptide consisting of two amino acids, said amino acids being natural amino acids, wherein at least one amino acids is a basic amino acid and a chloride-counterion, wherein the molar ratio of basic amino acid to chloride-ion is between 0.8 and 1.2.

In a preferred embodiment of this invention, the dipeptide is Xxx-Yyy or Yyy-Xxx, wherein Xxx is the basic amino acid and Yyy is another amino acid.

In a preferred embodiment of the present invention, the other amino acid Yyy is selected from cysteine/cystine (Cys) or tyrosine (Tyr).

In another preferred embodiment, the dipeptide is Xxx-Cys or Cys-Xxx, and wherein the dipeptide salt is in the form of (Xxx-Cys)₂ 2HCl or (Cys-Xxx)₂ 2HCl.

The basic amino acid is preferably selected from lysine (Lys), arginine (Arg) and histidine (His). The basic amino acid can be in the N-terminal or C-terminal position.

In a further preferred embodiment, the dipeptide salt is (Lys-Cys)₂ 2HCl according to formula I:

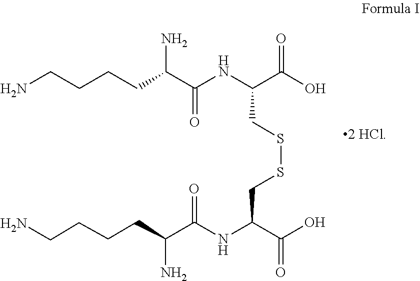 [SMILES: NCCCC[C@H](N)C(=O)N[C@@H](CSSC[C@H](NC(=O)[C@@H](N)CCCCN)C(=O)O)C(=O)O]

A “peptide” shall be understood as being a molecule comprising at least two amino acids covalently coupled to each other by alpha-peptide bonds (R¹—CO—NH—R²).

A “dipeptide” shall be understood as being a molecule comprising two amino acids covalently coupled to each other by an alpha-peptide-bond (R¹—CO—NH—R²).

An “amino acid”, in the context of the present invention, shall be understood as being a molecule comprising an amino functional group (—NH₂) and a carboxylic acid functional group (—COOH), along with a side-chain specific to the respective amino acid. In the context of the present invention, both alpha- and beta-amino acids are included. Preferred amino acids of the invention are alpha-amino acids, in particular the 20 “natural amino” acids including cystine as follows:

| | |
|---|---|
| Alanine | (Ala/A) |
| Arginine | (Arg/R) |
| Asparagine | (Asn/N) |
| Aspartic acid | (Asp/D) |
| Cysteine | (Cys/C) |
| Cystine | (Cyss/C2) |
| Glutamic acid | (Glu/E) |
| Glutamine | (Gln/Q) |
| Glycine | (Gly/G) |
| Histidine | (His/H) |
| Isoleucine | (Ile/I) |
| Leucine | (Leu/L) |
| Lysine | (Lys/K) |
| Methionine | (Met/M) |
| Phenylalanine | (Phe/F) |
| Proline | (Pro/P) |
| Serine | (Ser/S) |
| Threonine | (Thr/T) |
| Tryptophan | (Trp/W) |
| Tyrosine | (Tyr/Y) |
| Valine | (Val/V) |

In the context of the present invention, the expression “natural amino acids” shall be understood to include both the L-form and the D-form of the above listed 20 amino acids. The L-form, however, is preferred. In one embodiment, the term “amino acid” also includes analogues or derivatives of those amino acids.

A “free amino acid”, according to the invention (for instance “free cysteine”), is understood as being an amino acid having its amino and its (alpha-) carboxylic functional group in free form, i.e., not covalently bound to other molecules, e.g., an amino acid not forming a peptide bond. Free amino acids may also be present as salts or in hydrate form. When referring to an amino acid as a part of, or in, a dipeptide, this shall be understood as referring to that part of the respective dipeptide structure derived from the respective amino acid, according to the known mechanisms of biochemistry and peptide biosynthesis.

The expression “N-acylated”, with reference to a chemical compound, such as an amino acid, shall be understood as meaning that the N-acylated compound is modified by the addition of an acyl group to a nitrogen functional group of said compound. Preferably, the acyl group is added to the alpha-amino group of the amino acid.

The dipeptide salts can be in the form of solids (crystalline powders, agglomerates, etc.) or be provided in an aqueous solution. Concentrated stock solution should have a concentration of greater 25 mM, preferably greater 100 mM, most preferably greater 200 mM.

The dipeptide salts can also be used together with other commonly used dipeptides, that do not contain basic amino acids, such as Ala-Gln, Gly-Gln, Ala-Tyr, Gly-Tyr or Ala-Cys or (Ala-Cys)₂.

In the context of this invention, Cys-peptides forming a disulfide bond via oxidized cysteine residues, shall be described by (Xxx-Cys)₂ or (Cys-Xxx) 2. The peptides may also be present in hydrate form. Such disulfide bond mediated dimers of Cys-dipeptides, for instance (Xxx-Cys)₂, are still considered as a dipeptide in the sense of the invention.

Preferably, the composition has a pH-value at 25° C. of at least 5 or preferred of at least 6. In a preferred embodiment, the dipeptide salts are either in a reduced state (=free thiol) or oxidized state (=disulfide bonded), preferably in an oxidized state.

In preferred embodiments, the dipeptide is not N-acylated. N-acylation is known to improve heat stability of certain dipeptide; however, it has been found that N-acylated dipeptides may also lead to inferior viable cell density and viability.

The present invention is also directed to a cosmetic product, a nutritional supplement, nutrient solution for clinical nutrition, or a biological drug product formulation comprising the composition according to the present invention.

The cosmetic product may be a shampoo, conditioner, lotion, cream or other formulations used to treat skin or hair. Nutritional supplements may be in liquid form, such as syrups or shots, or in solid form, such as capsules, soft-gels, gummies. The compositions can also be part of nutrient solutions for clinical enteral or parenteral nutrition, e.g. part of an amino acid solution such as Aminoven (Fresenius Kabi). The compositions can also be part of a biological drug product formulation, which are preferably selected from an antibody or vaccine formulations.

Moreover, the present invention also refers to a cell or tissue culture medium.

Another subject of the present invention is directed to a cell or tissue culture medium comprising the composition according to the present invention, which further comprises at least one carbohydrate, at least one free amino acid, at least one inorganic salt, a buffering agent and/or at least one vitamin. In a particularly preferred embodiment, the culture medium comprises all of at least one carbohydrate, at least one free amino acid, at least one inorganic salt, a buffering agent and at least one vitamin.

In one embodiment of the invention, the culture medium does not contain a growth factor. In accordance with this embodiment, the dipeptide salt of the invention may be used instead of a growth factor for promoting growth and/or proliferation of the cells in culture. In another embodiment of the invention, the culture medium does not contain any lipids.

According to another embodiment of the invention, the culture medium is in liquid form, in form of a gel, a powder, a granulate, a pellet or in form of a tablet.

In preferred embodiments, the culture medium of the invention is a defined medium, or a serum-free medium. For example, the compositions of the invention may be supplemented to the CHOMACS CD medium of Miltenyi Biotech (Bergisch Gladbach, Germany), to the PowerCHO-2 CD medium available from LONZA (Basel, Switzerland), the Acti-CHO P medium of PAA (PAA Laboratories, Pasching, Austria), the Ex-Cell CD CHO medium available from SAFC, the SFM4CHO medium and the CDM4CHO medium of ThermoFisher (Waltham, USA). The dipeptides of the invention may also be supplemented to DMEM medium (Life Technologies Corp., Carlsbad, USA). The invention, however, is not limited to supplementation of the above media.

In other preferred embodiments, the culture medium is a liquid medium in 2-fold, 3-fold, 3.33-fold, 4-fold, 5-fold or 10-fold concentrated form (volume/volume), relative to the concentration of said medium in use. This allows preparation of a “ready-to-use” culture medium by simple dilution of the concentrated medium with the respective volume of sterile water. Such concentrated forms of the medium of the invention may also be used by addition of the same to a culture, e.g., in a fed-batch cultivation or perfusion process.

The cell culture medium (cell or tissue culture basal, feed or perfusion medium) of the present invention may preferably contain all nutrients required for sustained growth and product formation. Recipes for preparing culture media, in particular cell culture media, are well known to the person skilled in the art (see, e.g., Cell Culture Technology for Pharmaceutical and Cell-Based Therapies, Öztürk and Wei-Shou Hu eds., Taylor and Francis Group 2006). Various culture media are commercially available from various sources.

The culture media of the invention may preferably include a carbohydrate source. The main carbohydrate used in cell culture media is glucose, routinely supplemented at 5 to 25 mM. In addition, any hexose, such as galactose, fructose, or mannose or a combination may be used.

The culture medium typically may also include at least the essential amino acids (i.e., His, Ile, Leu, Lys, Met, Phe, Thr, Try, Val) as well as non-essential amino acids. A non-essential amino acid is typically included in the cell culture medium if the cell line is not capable of synthesizing the amino acid or if the cell line cannot produce sufficient quantities of the amino acid to support maximal growth. In addition, mammalian cells can also use glutamine as a major energy source. Glutamine is often included at higher concentrations than other amino acids (2-8 mM). However, as noted above, glutamine can spontaneously break down to form ammonia and certain cell lines produce ammonia faster, which is toxic.

The culture media of the invention may preferably comprise salts. Salts are added to the cell culture medium to maintain isotonic conditions and prevent osmotic imbalances. The osmolality of a culture medium of the invention is about 300 mOsm/kg, although many cell lines can tolerate an approximately 10 percent variation of this value or higher. The osmolality of some insect cell cultures tends to be higher than 300 mOsm/kg, and this may be 0.5 percent, 1 percent, 2 to 5 percent, 5-10 percent, 10-15 percent, 15-20 percent, 20-25 percent, 25-30 percent higher than 300 mOsm/kg. The most commonly used salts in cell culture medium include Na⁺, K⁺, Mg²⁺, Ca²⁺, Cl⁻, SO₄²⁻, PO₄³⁻, and HCO₃⁻ (e.g., CaCl₂), KCl, NaCl, NaHCO₃, Na₂HPO₄).

Other inorganic elements may be present in the culture medium. They include Mn, Cu, Zn, Mo, Va, Se, Fe, Ca, Mg, Si, and Ni. Many of these elements are involved in enzymatic activity. They may be provided in the form of salts such as CaCl₂), Fe(NO₃)₃, MgCl₂, MgSO₄, MnCl₂, NaCl, NaHCO₃, Na₂HPO₄, and ions of the trace elements, such as, selenium, vanadium and zinc. These inorganic salts and trace elements may be obtained commercially, for example from Sigma (Saint Louis, Missouri).

The culture media of the invention preferably comprise vitamins. Vitamins are typically used by cells as cofactors. The vitamin requirements of each cell line vary greatly, although generally extra vitamins are needed if the cell culture medium contains little or no serum or if the cells are grown at high density. Exemplary vitamins preferably present in culture media of the invention include biotin, choline chloride, folic acid, i-inositol, nicotinamide, D-Ca⁺⁺-pantothenate, pyridoxal, riboflavin, thiamine, pyridoxine, niacinamide, A, B₆, B₁₂, C, D₃, E, K, and p-aminobenzoic acid (PABA).

Culture media of the invention may also comprise serum. Serum is the supernatant of clotted blood. Serum components include attachment factors, micronutrients (e.g., trace elements), growth factors (e.g., hormones, proteases), and protective elements (e.g., antitoxins, antioxidants, antiproteases). Serum is available from a variety of animal sources including human, bovine or equine serum. When included in cell culture medium according to the invention, serum is typically added at a concentration of 5-10% (vol.). Preferred cell culture media are serum-free.

To promote cell growth in the absence or serum or in serum reduced media, one or more of the following polypeptides can be added to a cell culture medium of the invention: for example, fibroblast growth factor (FGF), including acidic FGF and basic FGF, insulin, insulin-like growth factor (IGF), epithelial growth factor (EGF), nerve growth factor (NGF), platelet-derived growth factor (PDGF), and transforming growth factor (TGF), including TGFalpha and TGFbeta, any cytokine, such as interleukins 1, 2, 6, granulocyte stimulating factor, leukocyte inhibitory factor (LIF), etc.

In other embodiments, the cell culture medium does not comprise polypeptides (i.e., peptides with more than 20 amino acids).

One or more lipids can also be added to a cell culture medium of the invention, such as linoleic acid, linolenic acid, arachidonic acid, palmitoleic acid, oleic acid, polyenoic acid, and/or fatty acids of 12, 14, 16, 18, 20, or 24 carbon atoms, each carbon atom branched or unbranched), phospholipids, lecithin (phosphatidylcholine), and cholesterol. One or more of these lipids can be included as supplements in serum-free media. Phosphatidic acid and lysophosphatidic acid stimulate the growth of certain anchorage-dependent cells, such as MDCK, mouse epithelial, and other kidney cell lines, while phosphatidylcholine, phosphatidylethanolamine, and phosphatidylinositol stimulate the growth of human fibroblasts in serum-free media. Ethanolamine and cholesterol have also been shown to promote the growth of certain cell lines. In certain embodiment, the cell culture medium does not contain a lipid.

One or more carrier proteins, such as bovine serum albumin (BSA) or transferrin, can also be added to the cell culture medium. Carrier proteins can help in the transport of certain nutrients or trace elements. BSA is typically used as a carrier of lipids, such as linoleic and oleic acids, which are insoluble in aqueous solution. In addition, BSA can also serve as a carrier for certain metals, such as Fe, Cu, and Ni. In protein-free formulations, non-animal derived substitutes for BSA, such as cyclodextrin, can be used as lipid carriers.

One or more attachment proteins, such as fibronectin, laminin, and pronectin, can also be added to a cell culture medium to help promote the attachment of anchorage-dependent cells to a substrate.

The cell culture medium can optionally include one or more buffering agents. Suitable buffering agents include, but are not limited to, N-[2-hydroxyethyl]-piperazine-N′-[2-ethanesulfonic acid] (HEPES), MOPS, MES, phosphate, bicarbonate and other buffering agents suitable for use in cell culture applications. A suitable buffering agent is one that provides buffering capacity without substantial cytotoxicity to the cells cultured. The selection of suitable buffering agents is within the ambit of ordinary skill in the art of cell culture.

Polyanionic or polycationic compounds may be added to the culture medium to prevent the cells from clumping and to promote growth of the cells in suspension.

In a preferred embodiment, the culture medium is in liquid form. The culture medium, however, can also be a solid medium, such as a gel-like medium, e.g. an agar-agar-, carrageen- or gelatine-containing medium (powders, aggregated powders, instantized powders etc.). Preferably, the culture medium is in sterile form.

The culture medium of the present invention can be in concentrated form. It may be, e.g., in 2- to 100-fold concentrated form, preferably in 2-fold, 3-fold, 3.33-fold, 4-fold, 5-fold, 10-fold, 20-fold, 50-fold or 100-fold (relative to a concentration that supports growth and product formation of the cells). Such concentrated culture media are helpful for preparing the culture medium for use by dilution of the concentrated culture medium with an aqueous solvent, such as water. Such concentrated culture media may be used in batch culture but are also advantageously used in fed-batch or continuous cultures, in which a concentrated nutrient composition is added to an ongoing cultivation of cells, e.g., to replenish nutrients consumed by the cells during culture.

In other embodiments of the invention, the culture medium is in dry form, e.g., in form of a dry powder, or in form of granules, or in form of pellets, or in form of tablets.

The present invention also relates to the use of a culture medium of the invention for culturing cells. Another aspect of the invention relates to the use of a culture medium of the invention for producing a cell culture product.

A preferred embodiment of the invention relates to the use of a culture medium according to the invention for culturing animal cells or plant cells, most preferred mammalian cells. In specific embodiments the cells to be cultured are CHO cells, COS cells, VERO cells, BHK cells, HEK cells, HELA cells, AE-1 cells, insect cells, fibroblast cells, muscle cells, nerve cells, stem cells, skin cells, endothelial cells and hybridoma cells. Preferred cells of the invention are CHO cells and hybridoma cells. Most preferred cells of the invention are CHO cells. Particularly preferred CHO cells of the invention are CHO DG44 and CHO DP12 cells.

Also included in the scope of the present invention is a method of culturing cells, said method comprising contacting said cells with a cell culture medium according to the invention. In one embodiment of the invention, the method of culturing cells comprises contacting the cell with a basal culture medium under conditions supporting the cultivation of the cell and supplementing the basal cell culture medium with a concentrated medium according to the present invention. In preferred embodiments, the basal culture medium is supplemented with the concentrated feed or medium on more than one day.

Another aspect of the invention relates to a method of producing a culture medium according to the invention, wherein said culture medium comprises a composition according to the invention.

Methods of producing a culture medium according to the invention comprise at least one step of adding the composition of the invention to the culture medium. Likewise, an aspect of the invention relates to the use of a composition of the invention for producing a cell culture medium.

Another aspect of the invention relates to a method of modifying a culture medium, wherein said modifying of said culture medium comprises addition of the composition of the invention to said culture medium.

Another aspect of the invention relates to a method of producing a liquid culture medium, said method comprising providing solid medium according to the invention, e.g., in form of a dry powder, or in form of granules, or in form of pellets, or in form of tablets; and dissolving said solid culture medium in an aqueous medium, such as water.

Another aspect of the invention relates to the use of a composition according to the invention in a culture medium for culturing cells. Another aspect of the invention relates to the use of a composition according to the invention for cell culture.

The invention also relates to methods of manufacturing a cell culture product comprising the steps of (i) providing a cell capable of producing said cell culture product; (ii) contacting said cell with a culture medium of the invention; and (iii) obtaining said cell culture product from said culture medium or from said cell. Likewise, the present invention relates to the use of a composition according to the invention for manufacturing a cell culture product.

In preferred methods, the cell culture product is a therapeutic protein, a diagnostic protein, a polysaccharide, such as heparin, an antibody, a monoclonal antibody, a growth factor, an interleukin, virus, virus-like particle or an enzyme.

Cultivation of cells, according to the invention can be performed in batch culture, in fed-batch culture or in continuous culture.

### EXAMPLES

### Materials:

TABLE 1
Materials used for in vitro viability assay

| Material | Supplier |
|---|---|
| CHO-K1 (hamster ovary cells) | DSMZ, Braunschweig (Germany) |
| Ham's F-12 Medium | Lonza Group AG, Basel |
| | (Switzerland) |
| Human Bone Marrow Stromal | STEMCELL technologies |
| Cells (MSC) | Germany GmbH, Köln (Germany) |
| MesenCult ™-ACF Plus | STEMCELL technologies |
| Culture Kit | Germany GmbH, Köln (Germany) |
| Gentamicin (50 mg/ml) | Thermo Fisher Scientific Inc., |
| | Waltham (USA) |
| Fetal bovine serum | Thermo Fisher Scientific Inc., |
| | Waltham (USA) |
| Pipet tips | Biozym Scientific GmbH, |
| | Hessisch Oldendorf (Germany) |
| 96 well plate, transparent, flat | Greiner Bio-One GmbH, |
| bottom | Kremsmünster (Austria) |
| Falcon tubes | Greiner Bio-One GmbH, |
| | Kremsmünster (Austria) |
| CombiTips | Eppendorf AG, Hamburg |
| | (Germany) |
| N,N′-di-L-lysyl-L-cystine | Evonik Operations GmbH, |
| dihydrochloride)/(Lys-Cys)₂ | Darmstadt (Germany) |
| 2 HCl | |
| N,N′-di-L-lysyl-L-cystine | Evonik Operations GmbH, |
| tetrahydrochloride/(Lys-Cys)₂ | Darmstadt (Germany) |
| 4 HCl | |
| CellTiter 96 ® AQueous Non- | Promega Corp., Madison (USA) |
| Radioactive Cell Proliferation | |
| Assay (MTS) | |

TABLE 2
Devices used for cytokine release assay.

| Device | Supplier |
|---|---|
| Safety cabinet HERA | Thermo Fisher Scientific GmbH, |
| Safe 2020 | Dreieich (Germany) |
| Heracell ™ 150i CO2 | Thermo Fisher Scientific GmbH, |
| Incubator | Dreieich (Germany) |
| Automatic cell counter | Thermo Fisher Scientific GmbH, |
| Countess | Dreieich (Germany) |
| Centrifuge 5415R | Eppendorf AG, Hamburg (Germany) |
| Vortex-Genie 2 | Scientific Industries Inc., Bohemia (USA) |
| TECAN Infinite 200 | Tecan Group Ltd., Männedorf |
| Pro | (Switzerland) |
| Microscope Primo Vert | Carl Zeiss AG, Oberkochen (Germany) |
| Analytical Balance | Sartorius AG, Göttingern (Germany) |
| Eppendorf Pipets | Eppendorf AG, Hamburg (Germany) |
| Dispenser | Eppendorf AG, Hamburg (Germany) |
| pH-Meter | Mettler-Toledo Inc., Greifensee |
| | (Switzerland) |

### Methods:

### Preparation of (Lys-Cys)₂ Salts

Peptide synthesis was performed with commonly used protective groups in solution. The raw product was purified by chromatographic methods and finally, different salt forms were prepared by ion exchange chromatography. A chloride free, inner salt (basic form), the dihydrochloride (two of four lysine amino groups protonated with 2 chloride ions as counterions) and a tetrachloride form (all four lysine amino groups protonated with 4 chloride ions as counterions) were obtained by subsequent freeze-drying.

Therefore, a raw solution of (Lys-Cys)₂ was demineralized via strong acidic IEX. Elution with Ammonia allows to generate the (Lys-Cys)₂ free base. Further purification was performed using adsorbing resin. Once free based was purified it was dryed by freeze-drying. Alternatively (Lys-Cys) 2 free base can be transformed into (Lys-Cys)₂·2 HCl salt using diluted HCl (pH=5). The (Lys-Cys)₂·2 HCl can be isolated by crystallization in EtOH and subsequently dried.

In order to verify and characterize the 2 HCl salt, a titration curve of (Lys-Cys)₂ free base using 0.1N HCl was performed. The pH jump confirmed the stoichiometry of 1 mol of (Lys-Cys)₂ free base for 2 moles of HCl. Reverse back-titration of (Lys-Cys)₂·2 HCl using 0.1N NaOH also confirmed the stoichiometry.

The (Lys-Cys)₂·4 HCl salt was produced and isolated by salification of the (Lys-Cys)₂ free base with an excess of HCl, using >4 equivalents of diluted HCl (pH=2.0). The 4 HCl salt was isolated by crystallization in butanol and subsequently drying.

### In Vitro Cytotoxicity Assay on Mesenchymal Stem Cells and on Chinese Hamster Ovary Cells (Subclone K1):

The assay was performed with human bone marrow stromal cells, mesenchymal stem cells (MSC) or Chinese hamster ovary cells (subclone K1), respectively. In a first step, the cells were seeded in a transparent 96-well cell culture plate and incubated for 24 h in a CO₂-Incubator (37° C., 5% CO₂, 95% humidity) at a cell density of 10.000 cells/well and in a final volume of 100 μl/well. After the resting time of 24 hours the supernatants were discarded, and prepared dipeptide test compounds were added to the cells in a final volume of 100 μl/well. The control was rested in medium without dipeptide.

All samples were dissolved directly in associated cell culture medium. pH values of the samples were adjusted to pH 7 in advance, to exclude an influence on the cell viability due to a potentially lower pH value.

Different salt forms of (Lys-Cys)₂ were tested in two different concentrations and each sample was tested in triplicates.

After an incubation for 24 hours in a CO₂-Incubator (37° C., 5% CO₂, 95% humidity), the assay reagent was prepared according to the manufacturers manual and 20 μl of the reagent was added to the wells to achieve a final volume of 120 μl/well.

As a result of the reduction of MTS into a formazan product by metabolically active cells, an absorbance signal of formazan could be measured at a wavelength of 490 nm in a multiplate reader. The signal is directly proportional to the number of living cells in culture.

### Example 1: Effects of Different Salt Forms of Cys-Peptides on the Viability of Cells

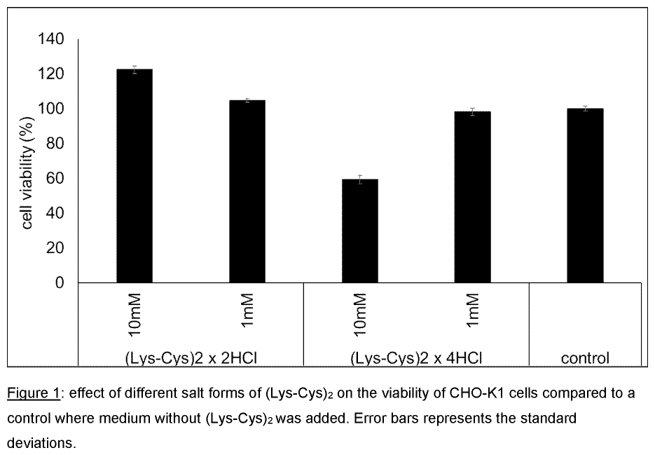

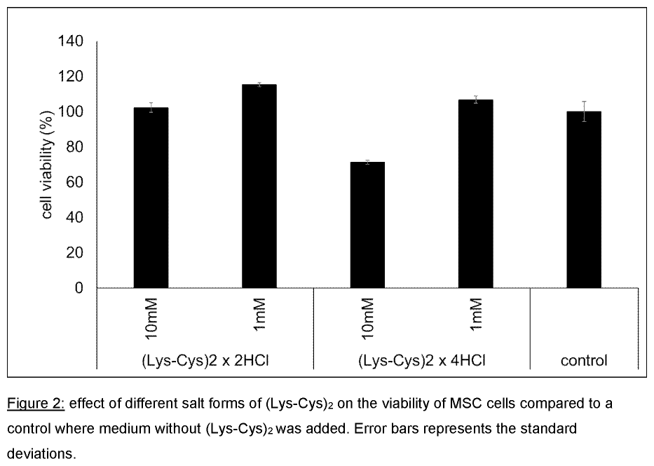

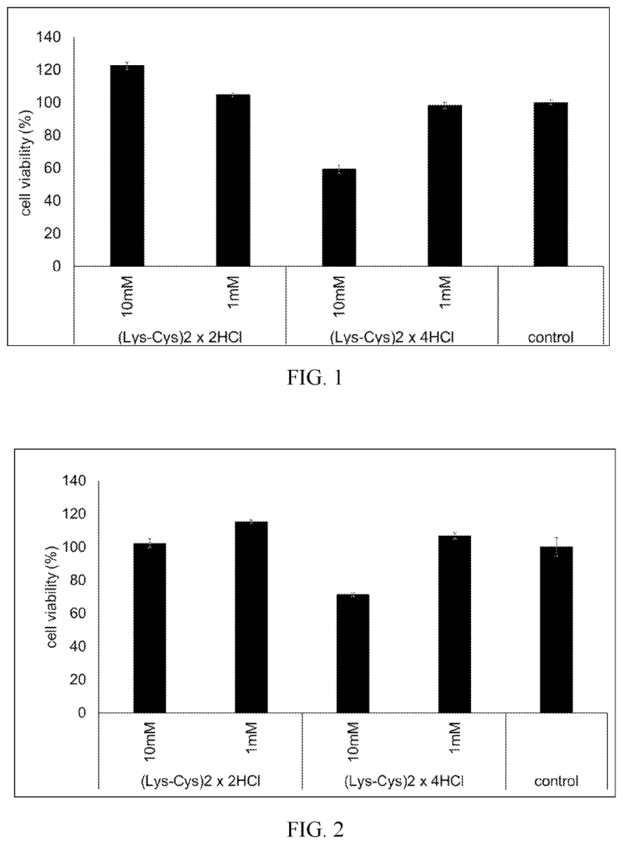

CHO-K1 as well as MSC cells were cultured for 24 hours before the addition of different (Lys-Cys)₂ salts. Cells were cultivated in presence of the di-hydrochloride form and the tetra-hydrochloride form of (Lys-Cys)₂, each peptide was applied in two different concentrations of 1 and 10 mM. After 24-hour cultivation with the dipeptides, the cell viability was assessed using the CellTiter 96® AQueous Non-Radioactive Cell Proliferation Assay (MTS). Results of these viability assays on CHO-K1 and MSC cells are shown in FIGS. 1 and 2.

FIG. 1 shows the effect of different salt forms of (Lys-Cys)₂ on the viability of CHO-K1 cells compared to a control where medium without (Lys-Cys)₂ was added. Error bars represents the standard deviations.

FIG. 2 shows the effect of different salt forms of (Lys-Cys)₂ on the viability of MSC cells compared to a control where medium without (Lys-Cys)₂ was added. Error bars represents the standard deviations.

Within these viability evaluations it was found that peptides containing basic amino acids, where the basic amino acid is fully protonated and two chloride counter ions are less biocompatible in high concentrations during cell culture than peptides containing basic amino acids, where the basic amino acid is only partially protonated and has one chloride counter ions (and that this is not caused by a pH effect). For example, in the case of (Lys-Cys)₂, the dihydrochloride (2 HCl) form could be provided in significantly higher concentration than the tetrahydrochloride (4 HCl) forms. In fact, while high concentration of the 2 HCl form improved cell viability, the 4 HCl form reduced cell viability at the same concentration.

### Example 2: Improved Storage Stability of Chloride Salts Vs the Basic Form

Storage stability tests at 25° C. and at 60° C. were conducted with the different salt forms of the (Lys-Cys) 2. Stability was measured with a HPLC method. Both, dihydrochloride and tetrahydrochloride salts were found to be significantly more stable than the chloride free, basic form.

1. A dipeptide salt, comprising:
a dipeptide consisting of two amino acids, said two amino acids being natural amino acids, wherein at least one amino acid is a basic amino acid and a chloride-counterion, wherein a molar ratio of said basic amino acid to chloride-ion is between 0.8 and 1.2.

2. The dipeptide salt according to claim 1, wherein the dipeptide is Xxx-Yyy or Yyy-Xxx, wherein Xxx is the basic amino acid and Yyy is another amino acid.

3. The dipeptide salt according to claim 1, wherein the other amino acid Yyy is selected from the group consisting of cysteine/cystine (Cys) and tyrosine (Tyr).

4. The dipeptide salt according to claim 1, wherein the dipeptide is Xxx-Cys or Cys-Xxx, and wherein the dipeptide salt is in the form of (Xxx-Cys)₂ 2HCl or (Cys-Xxx)₂ 2HCl.

5. The dipeptide salt according to claim 1, wherein the basic amino acid is selected from the group consisting of lysine (Lys), arginine (Arg) and histidine (His).

6. The dipeptide salt according to claim 1, wherein the dipeptide salt is (Lys-Cys)₂ 2HCl according to formula I:

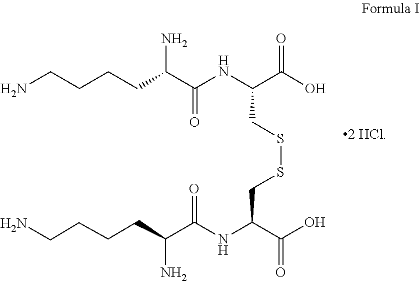 [SMILES: NCCCC[C@H](N)C(=O)N[C@@H](CSSC[C@H](NC(=O)[C@@H](N)CCCCN)C(=O)O)C(=O)O]

7. A cosmetic product, nutritional supplement, or nutrient solution for clinical nutrition or biological drug product formulation, comprising:
the dipeptide salt according to claim 1.

8. A cell culture medium, comprising:
a dipeptide salt according to claim 1, said culture medium further comprising at least one carbohydrate, and/or at least one additional free amino acid, and/or at least one inorganic salt, and/or a buffering agent and/or at least one vitamin.

9. The culture medium according to claim 8, wherein said culture medium is in liquid form, in form of a gel, a powder, a granulate, a pellet, or in the form of a tablet.

10. The culture medium according to claim 8, wherein said culture medium is in a 2- to 100-fold concentrated form relative to the concentration of the culture medium in use.

11. A method for culturing cells, the method comprising: culturing cells in a culture medium according to claim 8.

12. The method according to claim 11, wherein said cells are selected from the list consisting of CHO cells, COS cells, VERO cells, BHK cells, HEK cells, HELA cells, AE-1 cells, insect cells, fibroblast cells, muscle cells, nerve cells, stem cells, skin cells, endothelial cells, immune cells, and hybridoma cells.

13. A method of manufacturing a cell culture product, the method comprising:
providing a cell capable of producing said cell culture product;
contacting said cell with a culture medium of claim 8; and
obtaining said cell culture product from said culture medium or from said cell.

14. The method according to claim 11, wherein the culture medium is an aqueous stock or feed solution.

15. The method according to claim 12, wherein the immune cells are NK or T-cells.
